(19) United States
(12) Patent Application Publication
(10) Pub. No.: US 2023/0165535 A1
(43) Pub. Date: Jun. 1, 2023

(54) PHYSIOLOGICAL SIGNAL PROCESSING APPARATUS AND PHYSIOLOGICAL SIGNAL PROCESSING METHOD

(71) Applicant: NIHON KOHDEN CORPORATION, Tokyo (JP)

(72) Inventors: Toshiki AOKI, Tokorozawa-shi, Saitama (JP); Satoru TOGO, Tokorozawa-shi, Saitama (JP); Shinya OKUNO, Tokorozawa-shi, Saitama (JP)

(21) Appl. No.: 17/995,729

(22) PCT Filed: Mar. 29, 2021
(86) PCT No.: PCT/JP2021/013416

§ 371 Date: Oct. 7, 2022

(30) Foreign Application Priority Data
Apr. 9, 2020 (JP) 2020-070462

(51) Int. Cl. A61B 5/00; G16H 40/67

(52) U.S. Cl. CPC A61B 5/7225; A61B 5/7203; G16H 40/67

(57) ABSTRACT

A physiological signal processing apparatus for converting a physiological signal into sampling data having a predetermined frequency that is set according to the physiological signal includes an A/D converter and a computation unit. The A/D converter is configured to convert the physiological signal into high-speed sampling data by sampling the physiological signal at a frequency that is higher than the predetermined frequency. The computation unit is configured to convert the high-speed sampling data into sampling data having the predetermined frequency by setting time windows for the high-speed sampling data and calculating one representative value for each of the windows on the basis of high-speed sampling data in the window.

### TECHNICAL FIELD

The presently disclosed subject matter relates to a physiological signal processing apparatus, a physiological signal processing program, and a physiological signal processing method.

### BACKGROUND

Health care professionals decide on a condition change and an abnormality of a subject on the basis of a physiological signal detected from the subject.

Noise that is generated in the environment or the like of detection of a physiological signal by a sensor may be superimposed on a physiological signal depending on the environment or the like. For example, there may occur a case that during an MRI examination a partial pressure of carbon dioxide contained in exhaled air of a sensor-attached patient is used for judging whether the patient is breathing. In this case, random noise may be superimposed on a physiological signal due to a magnetic field generated by an MRI apparatus. Noise superimposed on a physiological signal may make it difficult for a health care professional to judge on the basis of the physiological signal.

Among techniques for eliminating noise from a physiological signal is one disclosed in the following Patent Literature 1. In this technique, exhalation sounds or the like detected consecutively are converted into time-series digital samples. Among the digital samples, a current sample group including three digital samples centered by a sample of attention and sample groups immediately before and after the current sample group in time are determined. If the gradient between any two digital samples of each sample group exceeds a threshold value and the value of the sample of attention is larger than center values of the respective sample groups immediately before and after the current sample group, it is inferred that the sample of attention is part of a noise spike. The value of the sample of attention is decreased by replacing it with the value of a point on a line that connects the above center values. In this manner, the noise is eliminated from the physiological signal.

### CITATION LIST

### Patent Literature

Patent Literature 1: US Patent Application Publication No. 2015-0199951

### SUMMARY

### Technical Problem

However, as the environment and situation of detection of a physiological signal diversify, noise can be superimposed on a physiological signal in such a manner as to vary in frequency depending on the environment. This makes it difficult to eliminate noise by a lowpass filter. Furthermore, the ratio of noise contained in digital data may become relatively large depending on the sampling frequency used in converting a noise-superimposed physiological signal into the digital data, making it difficult to eliminate the noise from the digital data. The above prior art technique cannot accommodate these problems.

The presently disclosed subject matter has been conceived to solve the above problems. An object of the presently disclosed subject matter is therefore to provide a physiological signal processing apparatus, a physiological signal processing program, and a physiological signal processing method capable of eliminating noise effectively from a physiological signal irrespective of the properties of the noise superimposed on the physiological signal.

### Solution to Problem

The above problems to be addressed by the presently disclosed subject matter can be solved by the following means.

A physiological signal processing apparatus for converting a physiological signal into sampling data having a predetermined frequency that is set according to the physiological signal, including an A/D converter which is configured to convert the physiological signal into high-speed sampling data by sampling the physiological signal at a frequency that is higher than the predetermined frequency; and a computation unit which is configured to convert the high-speed sampling data into sampling data having the predetermined frequency by setting time windows for the high-speed sampling data and calculating one representative value for each of the windows on the basis of high-speed sampling data in the window.

A control program of a physiological signal processing apparatus for converting a physiological signal into sampling data having a predetermined frequency that is set according to the physiological signal, the control program serving to cause a computer to execute the steps of:

(A) converting the physiological signal into high-speed sampling data by causing an A/D converter to sample the physiological signal at a frequency that is higher than the predetermined frequency; and

(B) converting the high-speed sampling data into sampling data having the predetermined frequency by setting time windows for the high-speed sampling data and calculating one representative value for each of the windows on the basis of high-speed sampling data in the window.

A physiological signal processing method that is performed by a physiological signal processing apparatus for converting a physiological signal into sampling data having a predetermined frequency that is set according to the physiological signal, including the steps of:

(A) converting the physiological signal into high-speed sampling data by sampling the physiological signal at a frequency that is higher than the predetermined frequency; and

(B) converting the high-speed sampling data into sampling data having the predetermined frequency by setting time windows for the high-speed sampling data and calculating one representative value for each of the windows on the basis of high-speed sampling data in the window.

### Effects of the Invention

A physiological signal is converted into sampling data having a predetermined frequency by converting the physiological signal into high-speed sampling data by sampling it at a frequency that is higher than the predetermined frequency that is set for the physiological signal (a predetermined frequency is set for each physiological signal) and then calculating one representative value for each of time windows that are set for the high-speed sampling data. In this manner, noise can be eliminated effectively from the physiological signal irrespective of the properties of the noise superimposed on the physiological signal.

### BRIEF DESCRIPTION OF DRAWINGS

FIG. 1 is a block diagram illustrating a hardware configuration of a physiological signal processing apparatus.

FIG. 2 is a block diagram illustrating functions of a signal generator.

FIG. 3 is a graph of a waveform of high-speed sampling data of a physiological signal on which no noise is superimposed.

FIG. 4 is a graph of a waveform of high-speed sampling data of a physiological signal on which noise is superimposed.

FIG. 5 is a graph of a waveform (comparative example) of sampling data, sampled at a predetermined frequency, of the physiological signal on which no noise is superimposed.

FIG. 6 is a graph of a waveform (comparative example) of sampling data, sampled at the predetermined frequency, of the physiological signal on which noise is superimposed.

FIG. 7 is a graph explaining time windows that are set for the high-speed sampling data shown in FIG. 4 of the physiological signal on which noise is superimposed.

FIG. 8 is a graph of sampling data sampled at the predetermined frequency and being based on representative values.

FIG. 9 is another block diagram illustrating functions of the signal generator.

FIG. 10 is a flowchart showing how the physiological signal processing apparatus operates.

### DESCRIPTION OF EMBODIMENTS

A physiological signal processing apparatus, a physiological signal processing program, and a physiological signal processing method according to an embodiment of the presently disclosed subject matter will be hereinafter described in detail with reference to the drawings. In the drawings, the same symbol will be used for the same element and redundant descriptions will be omitted. Ratios between dimensions used in the drawings may be exaggerated for convenience of description and be different from actual ones.

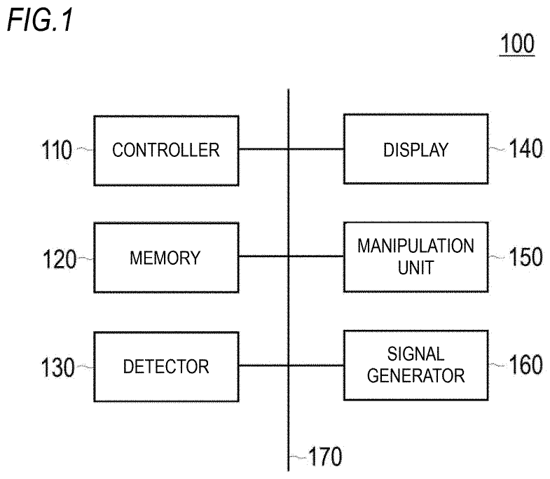

FIG. 1 is a block diagram illustrating a hardware configuration of a physiological signal processing apparatus 100.

The physiological signal processing apparatus 100 can include a controller 110, a memory 120, a detector 130, a display 140, a manipulation unit 150, and a signal generator 160. These components are connected to each other by a bus 170 so as to be able to communicate with each other.

The controller 110, which can be a CPU (central processing unit), controls the individual components of the physiological signal processing apparatus 100 and processes various kinds of data according to programs.

The memory 120 is, for example, a temporary storage device such as a RAM (random access memory), a secondary storage device such as a flash memory, or a non-transitory computer readable medium. For example, the RAM is used as a work area when the controller 110 runs programs. The flash memory or the non-transitory computer readable medium is stored with the programs according to which the controller 110 controls the individual components of the physiological signal processing apparatus 100 and processes various kinds of data. Various kinds of data are also stored in the flash memory or the non-transitory computer readable medium.

The detector 130 detects a physiological signal. For example, the detector 130 can detect a physiological signal in the form of an analog electrical signal. Physiological signals include, for example, partial pressure values of carbon dioxide in exhaled air (hereinafter referred to as “CO2 values”) detected by a CO2 sensor and an artery oxygen saturation detected by an SpO2 sensor. In the following, to simplify the description, descriptions will be made of a case that a physiological signal represents CO2 values.

The display 140 is, for example, a liquid crystal display which displays various kinds of information.

For example, the manipulation unit 150 is configured of a touch panel or various keys. The manipulation unit 150 is used for allowing a user to perform various kinds of manipulations.

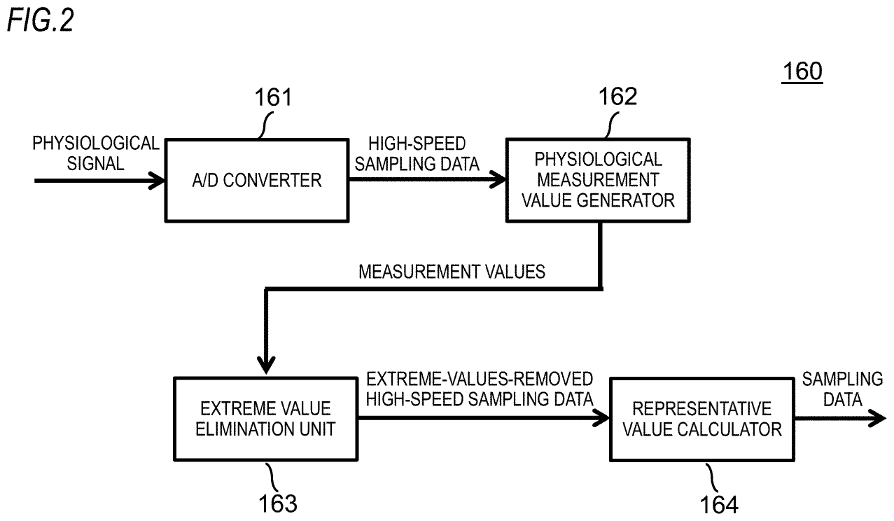

FIG. 2 is a block diagram illustrating functions of the signal generator 160.

The signal generator 160 functions as an A/D converter 161, a physiological measurement value generator 162, an extreme value elimination unit 163, and a representative value calculator 164. The physiological measurement value generator 162, the extreme value elimination unit 163, and the representative value calculator 164 can be configured using a CPU or the like. The physiological measurement value generator 162, the extreme value elimination unit 163, and the representative value calculator 164 configure a computation unit.

The A/D converter 161 converts a physiological signal into a high-speed sampling data by sampling the physiological signal at a frequency that is higher than a predetermined frequency. For example, the A/D converter 161 is implemented as a semiconductor chip that is mounted with electronic circuits. The predetermined frequency is set at a proper frequency in advance according to a physiological signal (more specifically, the frequency of a physiological signal) and stored in the memory 120. For example, in the case of a physiological signal representing CO2 values of exhaled air, the maximum frequency of a physiological signal is equal to about 2.5 Hz and the predetermined frequency is set at 15.6 Hz which is equal to about 6 times the frequency of the physiological signal. In the case of a physiological signal representing an artery oxygen saturation (SpO2) value detected by an SpO2 sensor, the maximum frequency of the physiological signal is equal to about 5 Hz and the predetermined frequency is set at 125 Hz which is equal to 25 times the frequency of the physiological signal.

The physiological measurement value generator 162 converts the high-speed sampling data into physiological signal measurement values such as CO2 values.

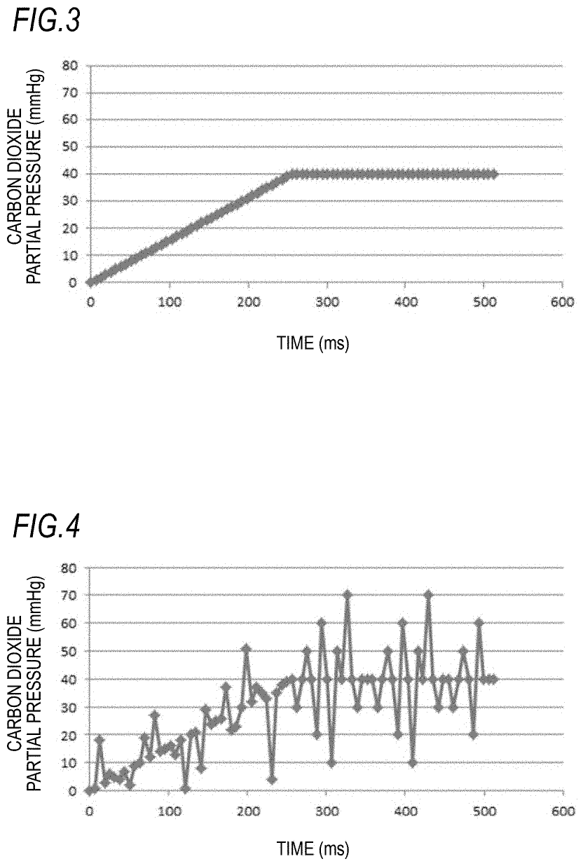

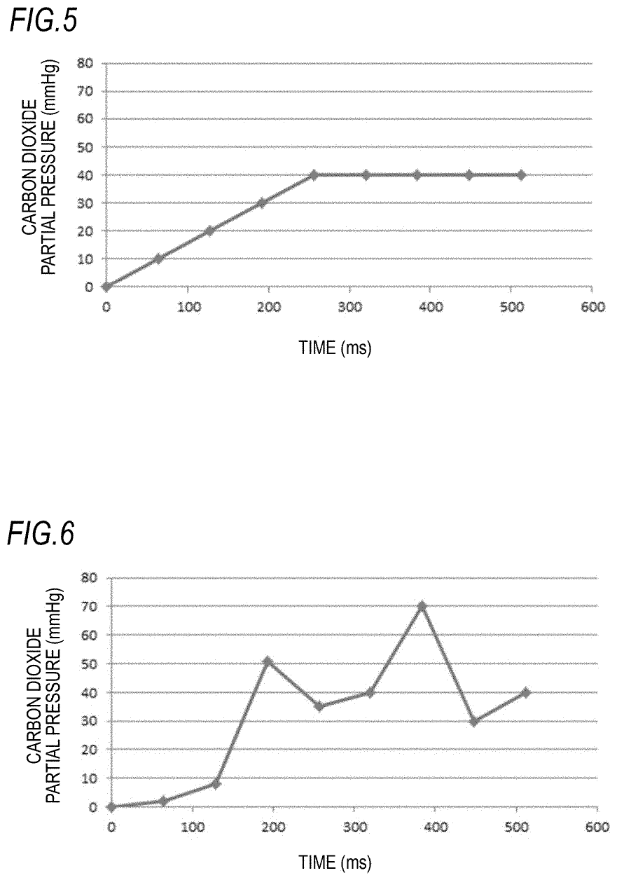

FIG. 3 is a graph of a waveform of high-speed sampling data of a physiological signal on which no noise is superimposed. FIG. 4 is a graph of a waveform of high-speed sampling data of a physiological signal on which noise is superimposed. FIG. 5 is a graph of a waveform (comparative example) of sampling data, sampled at a predetermined frequency, of the physiological signal on which no noise is superimposed. FIG. 6 is a graph of a waveform (comparative example) of sampling data, sampled at the predetermined frequency, of the physiological signal on which noise is superimposed. More specifically, the waveforms shown in FIGS. 3 to 6 are waveforms of physiological signal measurement values and are graphs in which the horizontal axis and the vertical axis represent time and the CO2 value, respectively.

As in the comparative example of FIG. 5, in the case where no noise is superimposed on a physiological signal, naturally no influence of noise appears in sampling data sampled at a predetermined frequency. However, as in the comparative example of FIG. 6, in the case where noise is superimposed on a physiological signal, remarkable influence of noise possibly can appear in sampling data sampled at a predetermined frequency. This phenomenon is considered to occur in such a manner that when random noise having a relatively high frequency is sampled at the predetermined frequency, noise signals are sampled depending on the sampling timing and the noise ratio in sampling data is made relatively large. Even in the case of the sampling data shown in the comparative example of FIG. 6, the noise frequency component can be eliminated using a lowpass filter before the sampling if the frequency of noise that may be superimposed on a physiological signal is known. However, where the frequency of noise that may be superimposed on a physiological signal cannot be determined in advance, it is difficult to eliminate the noise.

In the embodiment, where no noise is superimposed on a physiological signal as in the case of FIG. 3, naturally no influence of noise appears in high-speed sampling data. Where noise is superimposed on a physiological signal, as shown in FIG. 4 influence of noise appears in high-speed sampling data but the noise ratio in the high-speed sampling data is smaller than in the comparative example of FIG. 6, that is, the ratio of the physiological signal is relatively large. This is because the sampling at a frequency that is higher than the predetermined frequency can suppress the influence of the phenomenon that noise signals are sampled depending on the sampling timing.

The extreme value elimination unit 163 eliminates data that are larger than a predetermined upper limit threshold value or smaller than a predetermined lower limit threshold value or data whose variation rates are higher than a predetermined variation rate (these data are hereinafter referred to as “extreme values”) from the high-speed sampling data as converted into the physiological signal measurement values. The term “variation rate” means a temporal variation rate of the high-speed sampling data. The predetermined upper limit threshold value, the predetermined lower limit threshold value, and the predetermined variation rate can be set at proper values by experiments in view of the accuracy of the sampling data obtained by converting the physiological signal by the physiological signal processing apparatus 100. The extreme value elimination unit 163 may exclude only the extreme values that are larger than the predetermined upper limit threshold value. In this case, no predetermined lower limit threshold value is set. The extreme value elimination unit 163 may exclude only the extreme values that are smaller than the predetermined lower limit threshold value. In this case, no predetermined upper limit threshold value is set. In the extreme value elimination unit 163, different predetermined upper limit threshold values and different predetermined lower limit threshold values may be set for respective time windows (described later). As described later, the function of the extreme value elimination unit 163 can be omitted depending on representative values to be determined by the representative value calculator 164.

The extreme value elimination unit 163 may replace extreme values with other values instead of excluding the extreme values. For example, the other value can be a value obtained by adding the product of the above-mentioned predetermined variation rate and a time corresponding to the predetermined frequency to high-speed sampling data that was sampled immediately before sampling of high-speed sampling data having an extreme value. The other value may be a value that is smaller than or equal to the predetermined upper limit value and larger than or equal to the predetermined lower limit value or a value within a predetermined range of the predetermined upper limit value or the predetermined lower limit value.

The representative value calculator 164 sets time windows for the high-speed sampling data and converts the high-speed sampling data into sampling data having the predetermined frequency by calculating one representative value on the basis of high-speed sampling data in each window.

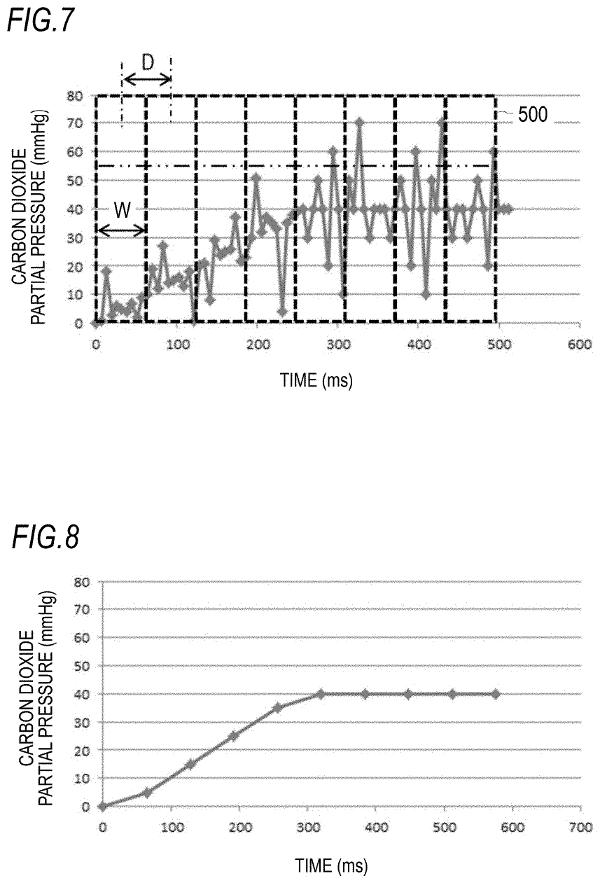

FIG. 7 is a graph explaining time windows 500 that are set for the high-speed sampling data shown in FIG. 4 of the physiological signal on which noise is superimposed. In FIG. 7, the above-mentioned predetermined upper limit value is indicated by a two-dot chain line. To simplify the description, high-speed sampling data that are larger than the predetermined upper limit value are also shown in FIG. 7 instead of being eliminated.

Windows 500 can be set so as to have a predetermined time width W and a predetermined time interval D. The predetermined time interval D is set at a time interval corresponding to the above-mentioned predetermined frequency. That is, where the physiological signal is CO2 values of exhaled air, as mentioned above the predetermined frequency is set at 15.6 Hz and the predetermined time interval D can be set at 64 ms accordingly. Whereas it is preferable that the predetermined time width W be set at the same value as the predetermined time interval D, the former may be set at a value that is different from the latter. Although it is preferable that the predetermined time interval D and the predetermined time width W be a fixed value(s) (e.g., 64 ms for all the windows 500), sampling data whose frequency is approximately equal to the predetermined frequency can be obtained even if they have small deviations (e.g., they include 63 ms). Where the predetermined time interval D and the predetermined time width W are a fixed length(s), representative values (described later) are calculated at the same interval and hence accurate sampling data can be calculated.

Each representative value can be determined to be one high-speed sampling data in a window 500 obtained after excluding extreme values or replacing extreme values with other values by the extreme value elimination unit 163. Each representative value may be a median value or an average value of high-speed sampling data in a window 500 remaining after excluding extreme values or replacing extreme values with other values by the extreme value elimination unit 163. Where each representative value is a median value or an average value of high-speed sampling data in the window 500, the extreme value elimination unit 163 need not always exclude extreme values or replace extreme values with other values. Each representative value may be an average value of plural data around a median value of high-speed sampling data in a window 500 remaining after excluding extreme values or replacing extreme values with other values by the extreme value elimination unit 163 (a median value and two values adjacent to the median value (three values in total)). That is, each representative value may be a value that is calculated from high-speed sampling data in a window 500 obtained by removing extreme values or replacing extreme values with other values by the extreme value elimination unit 163 and represents values of the high-speed sampling data in the window 500.

Each representative value can also be calculated on the basis of all or part of data obtained by sorting, by magnitude, data values of high-speed sampling data in a window 500 remaining after removing extreme values or replacing extreme values with other values by the extreme value elimination unit 163. Each representative value may also be calculated on the basis of all or part of data obtained by sorting, by magnitude, data values of high-speed sampling data in a window 500 obtained without removing extreme values or replacing extreme values with other values by the extreme value elimination unit 163 (i.e., the function of the extreme value elimination unit 163 is omitted). Each representative value can be a value obtained by weighted averaging performed on the basis of the order of sorted high-speed sampling data. A value may be obtained by weighted averaging in which each piece of all or part of high-speed sampling data is weighted more heavily as it is closer, in order, to a median value. The weighted averaging can be performed applying an FIR filter to high-speed sampling data.

The representative value calculator 164 can calculate each representative value by applying a digital filter to high-speed sampling data in a window 500. For example, the digital filter includes an FIR filter and an IIR filter. Whereas a weighted average is calculated by the FIR filter, the FIR filter includes one that calculates an average by setting its coefficients at 1.

The representative value calculator 164 may apply a digital filter to calculated representative values. The digital filter can be a lowpass filter. For example, an FIR filter or an IIR filter can be used as the digital filter.

FIG. 8 is a graph of sampling data sampled at the predetermined frequency and being based on representative values.

As shown in FIG. 8, the noise (see FIG. 4) superimposed on the physiological signal is eliminated by converting the physiological signal into the high-speed sampling data and calculating the representative values for the respective windows that are set for the high-speed sampling data.

The representative value calculator 164 sends a calculated representative value to the display 140 every time it is calculated. The representative value calculator 164 causes the display 140 to display a waveform on the basis of the sampling data of the physiological signal. The representative value calculator 164 may send an image of waveform formed of a physiological signal by further functioning as an image forming unit for forming an image of a waveform of a physiological signal on the basis of sampling data of a converted physiological signal.

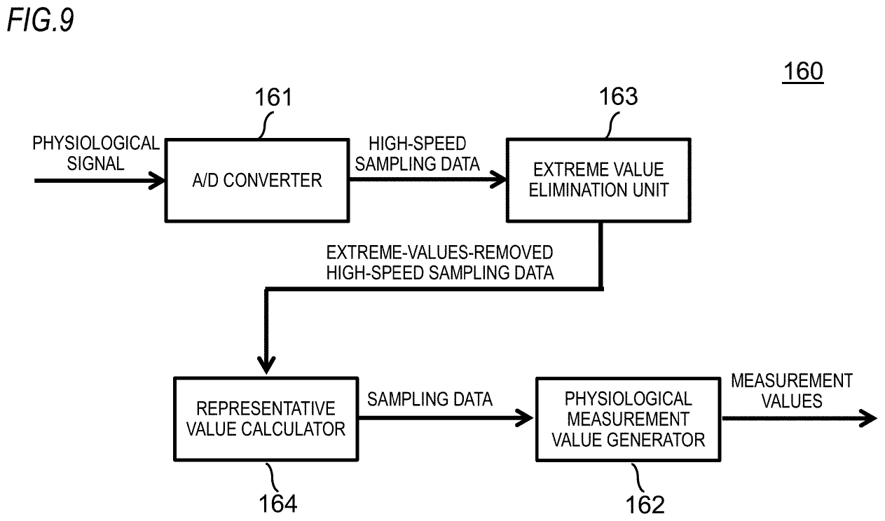

FIG. 9 is another example block diagram illustrating functions of the signal generator 160.

As show in FIG. 9, high-speed sampling data are not converted into measurement values of a physiological signal. Instead, a procedure can be followed that extreme values are excluded from the high-speed sampling data by the extreme value elimination unit 163, representative values are calculated by the representative value calculator 164, and the representative values are converted into physiological signal values by the physiological measurement value generator 162.

How the physiological signal processing apparatus 100 operates will be described.

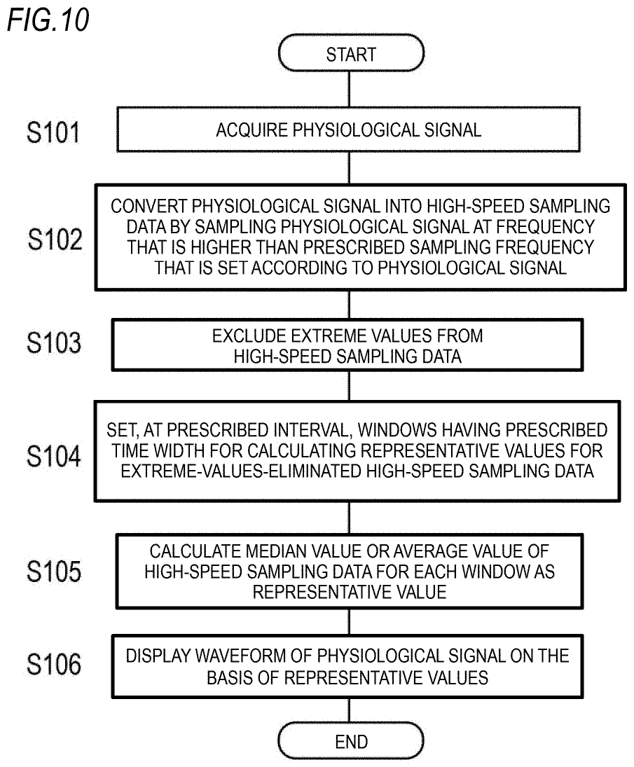

FIG. 10 is a flowchart showing how the physiological signal processing apparatus 100 operates. The signal generator 160 and/or the controller 110 can operate according to the programs so as to follow this flowchart. The following description will be made with an assumption that the signal generator 160 operates so as to follow this flowchart.

The signal generator 160 acquires a physiological signal from the detector 130 (S101).

The signal generator 160 converts the physiological signal into high-speed sampling data by sampling the physiological signal at a frequency that is higher than a predetermined sampling frequency that is set according to the physiological signal (S102).

The signal generator 160 excludes extreme values from the high-speed sampling data (S103), and sets, at a predetermined interval D, windows 500 having a predetermined time width W for calculating representative values for extreme-values-eliminated high-speed sampling data (S104).

The signal generator 160 calculates a median value or an average value of high-speed sampling data for each window 500 as a representative value (S105).

The signal generator 160 displays a waveform of the physiological signal on the display 140 on the basis of the representative values (S106).

The embodiment provides the following advantages.

A physiological signal is converted into sampling data having a predetermined frequency by converting the physiological signal into high-speed sampling data by sampling it at a frequency that is higher than the predetermined frequency that is set for the physiological signal (a predetermined frequency is set for each physiological signal) and then calculating one representative value for each of time windows that are set for the high-speed sampling data. In this manner, noise can be eliminated effectively from the physiological signal irrespective of the properties of the noise superimposed on the physiological signal.

One representative value is calculated for each of the windows set for the high-speed sampling data on the basis of the high-speed sampling data in the window after eliminating or replacing with other values extreme value data that are larger than a predetermined upper limit value or smaller than a lower limit threshold value. This measure makes it possible to eliminate noise easily and effectively.

One representative value is calculated on the basis of the high-speed sampling data in the window after eliminating or replacing with other values extreme value data whose variation rates are larger than a predetermined variation rate. This measure makes it possible to eliminate noise easily and effectively.

High-speed sampling data in each of the windows is sorted. One representative value is calculated on the basis of all or part of the sorted high-speed sampling data. This measure makes it possible to eliminate noise while lowering the influence of the noise on the physiological signal effectively.

Furthermore, a median value or an average value of the high-speed sampling data in the window is calculated as the one representative value. This measure makes it possible to lower the influence of noise on the physiological signal easily and effectively.

One representative value is calculated by applying a digital filter to the high-speed sampling data in the window. This measure makes it possible to lower the influence of noise on the physiological signal more easily.

One representative value is calculated by weighted averaging performed on the basis of the order of sorted high-speed sampling data. This measure makes it possible to lower the influence of noise on the physiological signal more effectively.

One of the high-speed sampling data in each window is calculated as a representative value after eliminating extreme values. This measure makes it possible to lower the influence of noise on the physiological signal more easily.

Still further, a digital filter is applied to the calculated representative value. This measure makes it possible to eliminate noise from the physiological signal more effectively.

Although the embodiment of the presently disclosed subject matter has been described in detail, the presently disclosed subject matter is not limited to the above-described embodiment.

For example, all or part of the functions that are realized by the programs in the above-described embodiment may be implemented by hardware such as circuits.

Furthermore, the physiological signal processing apparatus 100 may be composed of plural devices.

Still further, part of the steps of the above-described flowchart may be omitted and other steps may be added to the flowchart. Part of the steps may be executed at the same time and one step may be executed as plural divisional steps.

This application claims priority to Japanese Patent Application No. 2020-070462 filed on Apr. 9, 2020, the entire content of which is incorporated herein by reference.

### INDUSTRIAL APPLICABILITY

A physiological signal is converted into sampling data having a predetermined frequency by converting the physiological signal into high-speed sampling data by sampling it at a frequency that is higher than the predetermined frequency that is set for the physiological signal (a predetermined frequency is set for each physiological signal) and then calculating one representative value for each of time windows that are set for the high-speed sampling data. In this manner, noise can be eliminated effectively from the physiological signal irrespective of the properties of the noise superimposed on the physiological signal.

1. A physiological signal processing apparatus for converting a physiological signal into sampling data having a predetermined frequency that is set according to the physiological signal, comprising:
an A/D converter configured to convert the physiological signal into high-speed sampling data by sampling the physiological signal at a frequency that is higher than the predetermined frequency; and
a computation unit configured to convert the high-speed sampling data into sampling data having the predetermined frequency by setting time windows for the high-speed sampling data and calculating one representative value for each of the windows on the basis of high-speed sampling data in the window.

2. The physiological signal processing apparatus according to claim 1, wherein the computation unit is configured to calculate one representative value for each of the windows set for the high-speed sampling data on the basis of the high-speed sampling data in the window after eliminating or replacing with other values extreme value data that are larger than a predetermined upper limit value or smaller than a lower limit threshold value.

3. The physiological signal processing apparatus according to claim 1, wherein the computation unit is configured to calculate one representative value for each of the windows set for the high-speed sampling data on the basis of the high-speed sampling data in the window after eliminating or replacing with other values extreme value data whose variation rates are larger than a predetermined variation rate.

4. The physiological signal processing apparatus according to claim 1, wherein the computation unit is configured to sort high-speed sampling data in each of the windows and calculate one representative value on the basis of all or part of the sorted high-speed sampling data.

5. The physiological signal processing apparatus according to claim 2, wherein the computation unit is configured to calculate, as the one representative value, a median value or an average value of the high-speed sampling data in the window.

6. The physiological signal processing apparatus according to claim 2, wherein the computation unit is configured to calculate one representative value by applying a digital filter to the high-speed sampling data in the window.

7. The physiological signal processing apparatus according to claim 2, wherein the computation unit is configured to calculate one representative value by performing weighted averaging on the high-speed sampling data in the window.

8. The physiological signal processing apparatus according to claim 2, wherein the computation unit is configured to calculate, as a representative value, one of the high-speed sampling data in each window.

9. The physiological signal processing apparatus according to claim 1, wherein the computation unit is configured to apply a digital filter to the calculated representative value.

10. The physiological signal processing apparatus according to claim 1, wherein the windows have a fixed width corresponding to the predetermined frequency.

11. A non-transitory computer-readable medium configured to store a control program of a physiological signal processing apparatus for converting a physiological signal into sampling data having a predetermined frequency that is set according to the physiological signal, the control program serving to cause a computer to execute a processing including:
converting the physiological signal into high-speed sampling data by causing an A/D converter to sample the physiological signal at a frequency that is higher than the predetermined frequency; and
converting the high-speed sampling data into sampling data having the predetermined frequency by setting time windows for the high-speed sampling data and calculating one representative value for each of the windows on the basis of high-speed sampling data in the window.

12. A physiological signal processing method that is performed by a physiological signal processing apparatus for converting a physiological signal into sampling data having a predetermined frequency that is set according to the physiological signal, comprising:
converting the physiological signal into high-speed sampling data by sampling the physiological signal at a frequency that is higher than the predetermined frequency; and
converting the high-speed sampling data into sampling data having the predetermined frequency by setting time windows for the high-speed sampling data and calculating one representative value for each of the windows on the basis of high-speed sampling data in the window.
